(19) United States
(12) Patent Application Publication
(10) Pub. No.: US 2026/0026931 A1
(43) Pub. Date: Jan. 29, 2026

(54) PROSTHETIC HEART VALVE IMPLANT

(71) Applicant: Medtronic, Inc., Minneapolis, MN (US)

(72) Inventors: Anish S. Nigade, Santa Rosa, CA (US); Thuy Linh L. Dinh, Westminster, CA (US); Victoria T. Tien, Lakeville, MN (US); Mustafa E. Taskin, Durham, NC (US); Lijin Rajendran Nanu, Santa Rosa, CA (US); Alkindi Kibria, Irvine, CA (US); Fatemeh Fatemi Far, Richmond, CA (US); Shaefali M. Padiyar, Irvine, CA (US)

(21) Appl. No.: 19/229,367

(22) Filed: Jun. 5, 2025

Related U.S. Application Data
(60) Provisional application No. 63/676,212, filed on Jul. 26, 2024.

(51) Int. Cl. A61F 2/24

(52) U.S. Cl. CPC A61F 2/2418

(57) ABSTRACT

A heart valve prosthesis is positioned within an index valve prosthesis. The heart valve prosthesis includes an annular frame extending along a valve axis. The annular frame includes a plurality of frame members and is adjustable between a radially-collapsed configuration and a radially-expanded configuration. The annular frame includes an engagement region extending circumferentially around the valve axis. The engagement region includes at least one protrusion arm extending radially outwardly from the annular frame. The at least one protrusion arm includes a first arm end and a second arm end. The first arm end is attached to a frame member. The second arm end is spaced apart from the plurality of frame members and contacts a lacerated leaflet of the index valve prosthesis and moves the lacerated leaflet to a position that does not obstruct a coronary artery. Methods of implanting a heart valve prosthesis are provided.

### CROSS REFERENCE TO RELATED APPLICATIONS

This application claims the benefit of U.S. Provisional Patent Application No. 63/676,212, filed Jul. 26, 2024, the entire contents of which are incorporated herein by reference.

### FIELD

The present disclosure relates generally to a prosthetic heart valve assembly and, more particularly, to a heart valve prosthesis configured to be positioned within an index valve prosthesis.

### BACKGROUND

It is known to provide a prosthetic heart valve assembly for implanting a heart valve prosthesis within a target site of the vasculature of a patient. The heart valve prosthesis can be moved from a radially-contracted position to a radially-expanded position. However, positioning the heart valve prosthesis while avoiding obstruction of the coronary arteries can be difficult.

### SUMMARY

The following presents a simplified summary of the disclosure to provide a basic understanding of some aspects described in the detailed description.

In aspects, a heart valve prosthesis is configured to be positioned within an index valve prosthesis. The heart valve prosthesis comprises an annular frame extending along a valve axis between a first valve end of the heart valve prosthesis and a second valve end of the heart valve prosthesis. The annular frame comprises a plurality of frame members and is configured to be adjustable between a radially-collapsed configuration and a radially-expanded configuration. The annular frame comprises an engagement region extending circumferentially around the valve axis and comprises at least one protrusion arm extending radially outwardly from the annular frame. The at least one protrusion arm comprises a first arm end and an opposing second arm end. The first arm end is attached to a frame member of the plurality of frame members. The second arm end is spaced apart from the plurality of frame members and is configured to contact at least one lacerated leaflet of the index valve prosthesis and move the at least one lacerated leaflet to a position that does not obstruct a coronary artery.

In aspects, the at least one protrusion arm comprises a first protrusion arm comprising a first arm segment and a second arm segment. The first arm segment comprises the first arm end, and the second arm segment comprising the second arm end.

In aspects, an arm angle is defined between the first arm segment and the second arm segment, the arm angle within a range from about 60 degrees to about 120 degrees.

In aspects, the at least one protrusion arm comprises a second protrusion arm that is substantially identical in shape to the first protrusion arm. The second protrusion arm is attached to one of the plurality of frame members at substantially the same longitudinal location along the valve axis as the first protrusion arm and at a different circumferential location about the valve axis as the first protrusion arm.

In aspects, the second protrusion arm comprises a third arm segment and a fourth arm segment. The third arm segment comprises a first arm end attached to the frame member of the plurality of frame members, and the fourth arm segment comprises a fourth arm end opposite the third arm end.

In aspects, the second protrusion arm is substantially identical in shape to the first protrusion arm. The first protrusion arm and the second protrusion arm attached to one of the plurality of frame members at substantially the same longitudinal location along the valve axis and at substantially the same circumferential location about the valve axis.

In aspects, the second arm end and the fourth arm end comprise a rounded shape.

In aspects, the second arm end and the fourth arm end are each surrounded by a material comprising one or more of fabric or tissue.

In aspects, the at least one protrusion arm comprises a first arm segment, a second arm segment, and a third arm segment. The first arm segment comprises the first arm end, and the second arm segment and the third arm segment are attached to an end of the first arm segment.

In aspects, a heart valve prosthesis is configured to be positioned within an index valve prosthesis. The heart valve prosthesis comprises an annular frame extending along a valve axis between a first valve end of the heart valve prosthesis and a second valve end of the heart valve prosthesis. The annular frame comprises a plurality of frame members and is configured to be adjustable between a radially-collapsed configuration and a radially-expanded configuration. The annular frame comprises an engagement region extending circumferentially around the valve axis and comprising at least one protrusion arm extending radially outwardly from the annular frame. The at least one protrusion arm extends non-linearly between a first arm end and a second arm end. The first arm end is attached to a frame member of the plurality of frame members. The second arm end is spaced apart from the plurality of frame members and is configured to contact at least one lacerated leaflet of the index valve prosthesis and move the at least one lacerated leaflet to a position that does not obstruct a coronary artery.

In aspects, the at least one protrusion arm comprises a first protrusion arm comprising a first arm segment and a second arm segment, the first arm segment comprising the first arm end, and the second arm segment comprising the second arm end.

In aspects, an arm angle is defined between the first arm segment and the second arm segment, the arm angle within a range from about 60 degrees to about 120 degrees.

In aspects, the at least one protrusion arm comprises a second protrusion arm that is substantially identical in shape to the first protrusion arm. The second protrusion arm is attached to one of the plurality of frame members at substantially the same longitudinal location along the valve axis as the first protrusion arm and at a different circumferential location about the valve axis as the first protrusion arm.

In aspects, methods of implanting a heart valve prosthesis comprise lacerating index leaflets of an index valve prosthesis that is positioned in a native aortic annulus. Methods comprise delivering the heart valve prosthesis to an interior index lumen of the index valve prosthesis. Methods comprise deploying the heart valve prosthesis within the interior index lumen such that the heart valve prosthesis moves from a radially-compressed configuration to a radially-expanded configuration. The heart valve prosthesis comprises at least one protrusion arm extending radially outwardly from an annular frame. The at least one protrusion arm extends non-linearly between a first arm end, which is attached to a frame member of the plurality of frame members, and a second arm end. Methods comprise contacting the lacerated index leaflets of the index valve with the second arm end such that the lacerated index leaflets are moved to a position that does not obstruct a coronary artery.

In aspects, the second arm end comprises a rounded shape.

In aspects, the second arm end is surrounded by a material comprising one or more of fabric or tissue.

In aspects, the deploying the heart valve prosthesis comprises moving the at least one protrusion arm from a compressed position to a radially-expanded position.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of a transcatheter heart valve prosthesis in accordance with aspects of the disclosure;

FIG. 2 illustrates a top-down view of the transcatheter heart valve prosthesis in accordance with aspects of the disclosure;

FIG. 3 illustrates a side view of a delivery assembly for delivering the transcatheter heart valve prosthesis in accordance with aspects of the disclosure;

FIG. 4 illustrates a side view of the delivery assembly for delivering the transcatheter heart valve prosthesis in accordance with aspects of the disclosure;

FIG. 5 illustrates an introducer sheath in accordance with aspects of the disclosure;

FIG. 6 illustrates an introducer sheath in accordance with aspects of the disclosure;

FIG. 7 schematically illustrates a side view of an index transcatheter heart valve prosthesis positioned at a treatment site in accordance with aspects of the disclosure;

FIG. 8 illustrates an example of a transcatheter heart valve prosthesis in accordance with aspects of the disclosure;

FIG. 9 illustrates an example of deploying the transcatheter heart valve prosthesis from a capsule in accordance with aspects of the disclosure;

FIG. 10 illustrates an example of deploying the transcatheter heart valve prosthesis from a capsule in accordance with aspects of the disclosure;

FIG. 11 illustrates an example of deploying the transcatheter heart valve prosthesis from a capsule in accordance with aspects of the disclosure;

FIG. 12 illustrates an example of a transcatheter heart valve prosthesis in accordance with aspects of the disclosure;

FIG. 13 illustrates an example of deploying the transcatheter heart valve prosthesis from a capsule in accordance with aspects of the disclosure;

FIG. 14 illustrates an example of deploying the transcatheter heart valve prosthesis from a capsule in accordance with aspects of the disclosure;

FIG. 15 illustrates an example of a transcatheter heart valve prosthesis in accordance with aspects of the disclosure;

FIG. 16 illustrates an example of deploying the transcatheter heart valve prosthesis in accordance with aspects of the disclosure;

FIG. 17 illustrates an example of deploying the transcatheter heart valve prosthesis in accordance with aspects of the disclosure;

FIG. 18 illustrates an example of deploying the transcatheter heart valve prosthesis within the index transcatheter heart valve prosthesis in accordance with aspects of the disclosure; and

FIG. 19 illustrates an example of a transcatheter heart valve prosthesis in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The words “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

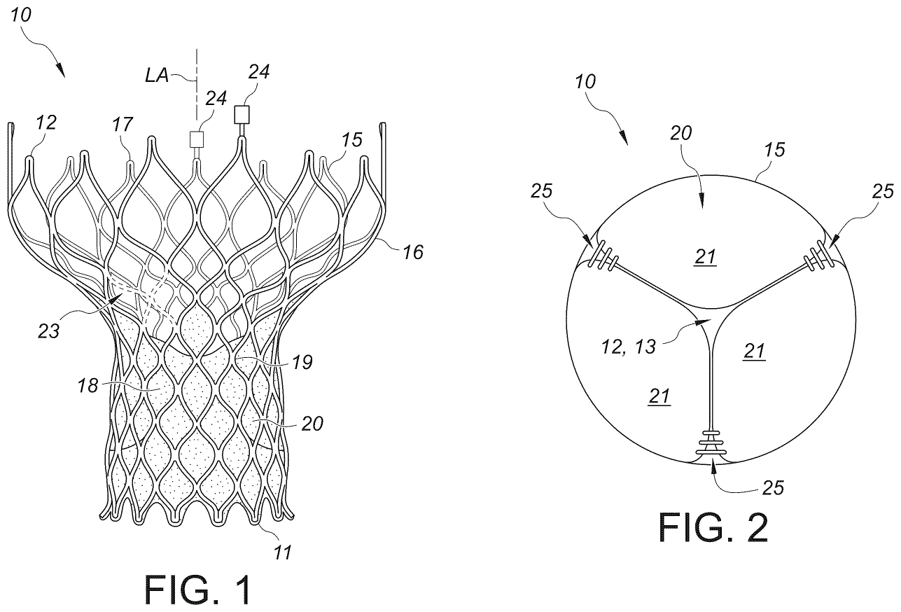

FIGS. 1 and 2 illustrate an example transcatheter heart valve prosthesis 10. The delivery assemblies described herein may be used with the transcatheter heart valve prosthesis 10 and/or other transcatheter heart valve prostheses. The transcatheter heart valve prosthesis 10 is illustrated to facilitate description of the disclosure. The following description of the transcatheter heart valve prosthesis 10 is merely exemplary in nature and is not intended to limit the invention or the application and uses of the invention.

FIGS. 1 and 2 illustrate a side view and a top/end view, respectively, of the transcatheter heart valve prosthesis 10. The transcatheter heart valve prosthesis 10 includes a radially-expandable frame or stent 15 and a prosthetic valve 20. The frame 15 of the transcatheter heart valve prosthesis 10 supports the prosthetic valve 20 within an interior of the frame 15. In the example transcatheter heart valve prosthesis 10 shown in FIGS. 1 and 2, the frame 15 is self-expandable. However, this is not meant to be limiting, and the frame 15 can be balloon-expandable or mechanically expandable in other embodiments. In some embodiments, the transcatheter heart valve prosthesis 10 may be delivered to and implanted at a treatment site within a patient to replace any of an aortic valve, a pulmonic valve, a mitral valve, and or a tricuspid valve. The valve to be replaced may be a native valve or a previously-implanted prosthetic valve, such as a failed surgical replacement valve or a failed transcatheter valve.

The prosthetic valve 20 includes at least one leaflet 21 disposed within and secured to the frame 15. In the embodiment shown in FIGS. 1 and 2, the prosthetic valve 20 includes exactly three leaflets 21, as shown in FIG. 2. However, this is not meant to be limiting, as the prosthetic valve 20 may include more or fewer leaflets 21. The valve leaflets 21 open and close to regulate flow through the transcatheter heart valve prosthesis 10.

As shown in FIG. 1, the transcatheter heart valve prosthesis 10 includes an inflow end 11 and an outflow end 12. The prosthetic leaflets 21 are attached to the frame 15 at commissures 25 such that when pressure at the inflow end 11 exceeds pressure at the outflow end 12, the prosthetic leaflets 21 open to allow blood flow through the heart valve prosthesis 10 from the inflow end 11 to the outflow end 12. When the pressure at the outflow end 12 exceeds pressure at the inflow end 11, the prosthetic leaflets 21 close to prevent blood flow from the outflow end 12 to the inflow end 11. Accordingly, the at least one leaflet (e.g., the prosthetic leaflets 21) can be attached to the plurality of struts 16, for example, by being directly attached to the plurality of struts 16 at the commissures 25, or by being indirectly attached to the plurality of struts 16, for example, by being attached to a skirt, a commissure bracket, or other structure (e.g., mechanical actuator) that is attached to the plurality of struts 16.

In aspects, the heart valve prosthesis 10 can comprise one or more attachment members 24 (e.g., paddles) positioned at an end, for example, the inflow end 11 or the outflow end 12. The attachment members 24 can be received within pockets of a spindle 38 (e.g., illustrated in FIG. 4), such that the spindle 38 and the attachment members 24 can interact to facilitate loading of the transcatheter heart valve prosthesis 10 and, in aspects, allow for possible recapture of the transcatheter heart valve prosthesis 10 during the deployment process. In aspects, and as described below, the attachment members 24 can be attached to either of the inflow end 11 or the outflow end 12. As such, the spindle 38 is configured to be positioned to receive the attachment members 24 either (1) adjacent to the inflow end 11 when the attachment members 24 are attached to the inflow end 11, or (2) adjacent to the outflow end 12 when the attachment members 24 are attached to the outflow end 12. Accordingly, the position of the attachment members 24 illustrated in FIG. 1 is merely exemplary.

The frame 15 of the transcatheter heart valve prosthesis 10 further includes a plurality of struts 16 that are arranged to form a plurality of openings or cells 18 arranged circumferentially around a longitudinal axis LA of the transcatheter heart valve prosthesis 10 and longitudinally to form a tubular structure defining a central lumen of the transcatheter heart valve prosthesis 10. For example, the frame 15 can extend along the longitudinal axis LA between the inflow end 11 and the outflow end 12. The frame 15 is configured to secure the prosthetic valve 20 within the central lumen of the frame 15 and to secure the transcatheter heart valve prosthesis 10 in place in the vasculature of the patient. The struts 16 are defined herein as the elongated wire segments of the frame 15. Struts 16 come together to form crowns 17 or nodes 19, as can be seen in FIG. 1. The frame 15 of the heart valve prosthesis 10 includes a plurality of cells 18 defined as the spaces between the plurality of crowns 17, the plurality of nodes 19, and the plurality of struts 16. The frame 15, and, thus, the plurality of struts 16, can be adjustable between a radially-collapsed position and a radially-expanded position.

In the example embodiment shown in FIG. 1, the plurality of cells 18 may be diamond-shaped. In the example embodiment shown, the plurality of cells includes a plurality of first cells 18 and, in aspects, access cells (e.g., an access cell 23). In particular, the access cells may be larger than the first cells 18 and can provide access to one or more coronary arteries when the transcatheter heart valve prosthesis 10 is implanted in the patient. FIG. 1 illustrates an example of an access cell 23, with the struts 16 at the access cell 23 illustrated with dashed lines to show that the struts 16 may not be present at the access cell 23, thus allowing for the access cell 23 to be larger than the first cells 18. The access cells can have an enlarged area relative or compared to the first cells 18. In some embodiments the transcatheter heart valve prosthesis 10 may include an outer skirt extending circumferentially around an outer circumference of the stent 15 at or near the inflow end 11 to prevent paravalvular leakage of blood around the outside of the transcatheter heart valve prosthesis 10 once implanted in the patient.

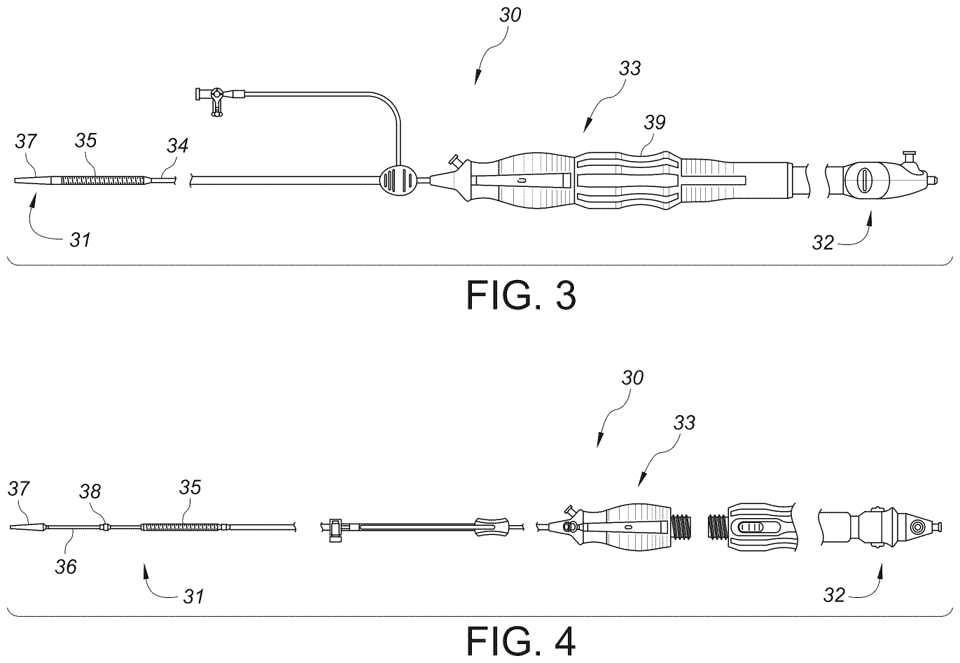

FIGS. 3 and 4 show schematically side views of a transcatheter heart valve delivery assembly 30 (e.g., “delivery assembly”) for delivering and deploying a transcatheter heart valve prosthesis (e.g., transcatheter heart valve prosthesis 10) according to embodiments hereof. One skilled in the art will realize that FIGS. 3 and 4 illustrate one example of a delivery assembly 30 and that components illustrated in FIGS. 3 and 4 may be removed and/or additional components may be added. The delivery assembly 30 includes a distal end 31, a proximal end 32, and a handle 33. The handle 33 enables a physician to manipulate a distal portion of the delivery assembly 30 and includes actuators for moving parts of the delivery assembly 30 relative to other parts. In the delivery assembly 30, an outer shaft 34 is coupled to an actuator 39 of the handle 33 for moving the outer shaft 34 relative to an inner shaft 36.

A distal portion of the outer shaft 34, referred to as a capsule 35, is configured to surround a transcatheter heart valve prosthesis (e.g., transcatheter heart valve prosthesis 10) during delivery to the treatment site (e.g., a native heart valve) and is retracted from the transcatheter heart valve prosthesis to expose the transcatheter heart valve prosthesis such that it self-expands (in self-expanding embodiments). In this way, the capsule 35 is in frictional engagement with the heart valve prosthesis 10. The inner shaft 36 can be coupled to the handle 33 (e.g., by being directly connected and in contact with the handle 33, or by being indirectly connected to the handle 33 with intermediate structures between the inner shaft 36 and the handle 33) and movement of the handle 33 can translate to movement of the inner shaft 36 and a distal tip or nosecone 37 coupled to a distal end of the inner shaft 36. The inner shaft 36 and distal tip or nosecone 37 may also be translated relative to the outer shaft 34 and the handle 33 via a tip retractor. In the embodiment shown, the inner shaft 36 includes a retainer or spindle 38 for receiving the paddles (e.g., attachment members 24) of the transcatheter heart valve prosthesis 10.

When the actuator 39 is actuated, the actuator 39 moves the outer shaft 34 and the capsule 35 relative to the inner shaft 36, as shown in FIG. 4. As known to those skilled in the art, when the delivery assembly 30 is in position such that the transcatheter heart valve prosthesis 10 is at the desired position at the treatment site in the patient's vasculature, the actuator 39 is actuated (e.g., rotated) to move the capsule 35 relative to the inner shaft 36 and the transcatheter heart valve prosthesis 10 disposed between the inner shaft 36 and the capsule 35, thereby enabling the transcatheter heart valve prosthesis 10 to deploy via self-expansion at the treatment site and release from the retainer 38, as shown in FIG. 4 (without showing the transcatheter heart valve prosthesis 10).

Minimally invasive percutaneous interventional procedures, including endovascular procedures, require access to the venous or arterial system. In general, it is desirable to make the smallest incision point with the shortest tissue contact time when entering the body. Small incisions and short tissue contact time generally lead to improved patient outcomes, less complications, and less trauma to the vessels or organs being accessed, as well as less trauma to the skin and tissue through which the access point is created. Access is required for various medical procedures that deliver or implant structural elements (such as heart valves, heart valve repair devices, occluders, grafts, electrical stimulators, leads, etc.) percutaneously. Some procedures employ relatively large devices that require relatively large sheaths to deliver the devices to the intended site within the body. With such procedures, access site trauma can occur, often resulting in vessel damage, excessive bleeding, increased case time, increased risk of infection, and increased hospitalization time. To reduce access trauma, physicians try to use the smallest devices possible and place the smallest sheath size. This can be problematic, however, if during the procedure the physician discovers a larger device is needed. This leads to a need to upsize the sheath, which is a lengthy procedure and leads to increased risk to the patient. Expandable sheaths can be expanded within the body and thus do not require removal to upsize.

Expandable sheath designs may be regionally or locally expansive to selectively and temporarily expand when the device is passing through a region of the sheath and to retract or recover when the device is not passing or has already passed through the sheath. Embodiments disclosed herein may be employed with an expandable introducer sheath that may solve these and other issues that contribute to vascular trauma. The expandable introducer sheath is described with respect to percutaneous access for transcatheter heart valve repair or replacement, and it should be understood that one or more features of the expandable introducer sheath may be employed alone or in combination for other medical procedures requiring percutaneous access, including but not limited to placement of stents, angioplasty, removal of arterial or venous calcification, and pre-dilatation or post-dilatation.

Various embodiments disclosed herein may include an introducer sheath that has a selectively expandable diameter to allow for the passage of a relatively larger device therethrough and further is configured to return to its original diameter upon passage of the device. The various embodiments may reduce damage to surrounding tissues by reducing contact with those tissues and by eliminating the need to exchange sheaths of different sizes. As a result, these embodiments can reduce procedure time, vascular trauma, bleeding, and the resulting risk of infection and other complications.

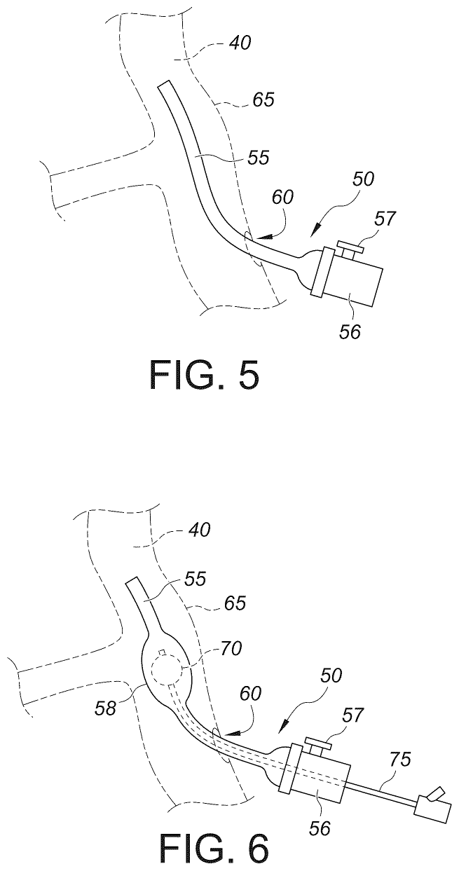

FIGS. 5 and 6 depict one embodiment of an introducer sheath 50 positioned through an incision 60 in the skin 65 of a patient and into a vessel 40 of a patient. The sheath 50 has a tubular shaft 55 and a proximal hub 56 with a hemostatic seal and a luer lock 57. FIG. 5 shows the sheath 50 positioned in the vessel 40 in its normal, unexpanded state, while FIG. 6 shows the sheath 50 positioned in the vessel 40 with a delivery device 75 delivering another device 70 that is being advanced through the sheath 50 such that the tubular shaft 55 expands or deforms at the location where the device 70 is passing through. The shaft 55 expands at expanded region 58 when the device 70 passes through and then retracts or recovers to its original diameter after the device 70 moves past or is removed from the shaft 55. Thus, the tubular shaft 55 is configured to be expandable and retractable.

In certain embodiments, the expandability of the shaft 55 (and any shaft described according to any embodiment set forth herein) is achieved via the elasticity of the shaft 55, which can result in the shaft 55 being either self-expandable or self-expanding or mechanically expandable or mechanically expanding. For purposes of this application, self-expandable means that the shaft 55 is configured to expand to a predetermined or nominal diameter automatically (without any type of actuation, mechanical or otherwise). Further, for purposes of this application, mechanically expandable means that the shaft 55 is configured to expand when a positionable medical device is positioned through the shaft 55. That is, the device itself that is being passed through the shaft 55 causes the expansion of the shaft 55, as depicted in FIG. 6. Alternatively, the expandable characteristics of the shaft 55 can be caused by something other than elasticity.

After passage of the device, the shaft 55 is configured to be contractable, retractable, or recoverable to its original, unexpanded state as depicted in FIG. 5. The retractability can be, in certain embodiments, achieved by the elasticity of the shaft 55, which can result in the shaft 55 being either self-retractable or self-retracting, self-recoverable, or self-contractable, or mechanically retractable or mechanically retracting, mechanically recoverable, or mechanically contractable. For purposes of this application, self-retractable means that the shaft 55 is configured to retract to a predetermined or nominal diameter automatically (without any type of actuation, mechanical or otherwise). Further, for purposes of this application, mechanically retractable means that the shaft 55 is configured to retract when a device or component is used to cause the shaft 55 to retract or recover. Alternatively, the retractable characteristics of the shaft 55 can be caused by something other than elasticity.

For purposes of this application, any device that can be positioned through an introducer sheath according to any embodiment disclosed or contemplated herein can be referred to as a positionable medical device or insertable medical device. Such devices include guidewires, dilators, delivery devices (for delivery and/or placement of structural elements such as heart valves, heart valve repair devices, occluders, grafts, electrical stimulators, leads, etc.), guide catheters, guiding sheaths, diagnostic catheters, stent delivery systems, balloon catheters, and other known vascular devices. Other devices can include non-vascular devices such as scopes and other common surgical instruments. Further, the introducer sheath is configured to receive tissues or organs. Thus, as one non-limiting example, the introducer sheath 50 is described as being an expandable introducer sheath 50 for introduction of a delivery assembly 30 including a transcatheter heart valve prosthesis 10.

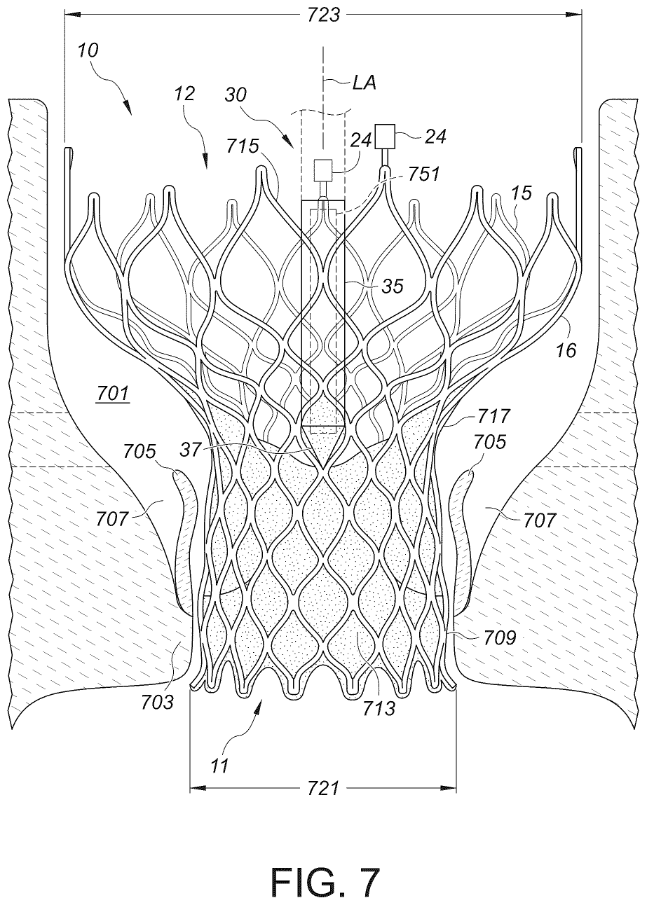

FIG. 7 illustrates the heart valve prosthesis 10 at a treatment site 701 within a patient's vasculature. In aspects, the treatment site 701 can comprise a location of a native aortic annulus (hereinafter “annulus”) 703 of a native heart valve, for example, the annulus of a patient's left ventricle. The treatment site 701 can comprise one or more native valve leaflets 705 and corresponding native sinuses 707. In some instances, paravalvular leakage can occur when blood travels through a gap 709 around the outside of the transcatheter heart valve prosthesis 10, with the gap 709 formed between the transcatheter heart valve prosthesis 10 and the annulus 703. To avoid paravalvular leakage, the heart valve prosthesis 10 can be radially expanded such that an outer radial surface of the heart valve prosthesis 10 can contact the annulus 703 and/or the native valve leaflets 705, thus reducing or eliminating the gap 709 and causing the blood to flow through the central lumen of the heart valve prosthesis 10. The heart valve prosthesis 10 can comprise the frame 15, which can comprise an asymmetric hourglass shape with a first section 713 at the inflow end 11, a second section 715 at the outflow end 12, and a waist section 717 positioned between the first section 713 and the second section 715. In aspects, the first section 713 can comprise a first diameter 721 and the second section 715 can comprise a second diameter 723, with the second diameter 723 greater than the first diameter 721. Additionally, in some embodiments the transcatheter heart valve prosthesis 10 may include an outer skirt extending circumferentially around an outer circumference of the frame 15 at or near the inflow end 11 to prevent paravalvular leakage of blood around the outside of the transcatheter heart valve prosthesis 10 once implanted in the patient.

In aspects, the heart valve prosthesis 10 can function as an index valve prosthesis (e.g., a previously-implanted heart valve prosthesis), such that another heart valve prosthesis can be delivered and deployed within the heart valve prosthesis 10. As illustrated in FIG. 7, the delivery assembly 30 can deliver a heart valve prosthesis 751 to an interior of the heart valve prosthesis 10. The delivery assembly 30 can comprise the capsule 35 and the distal tip 37, with the heart valve prosthesis 751 in a radially-compressed position and loaded within the capsule 35. In aspects, the heart valve prosthesis 751 can have some similarities in structure to the heart valve prosthesis 10 described herein, though the heart valve prosthesis 751 can function as a new or redo heart valve prosthesis that is deployed within the index, or previously-implanted, heart valve prosthesis 10.

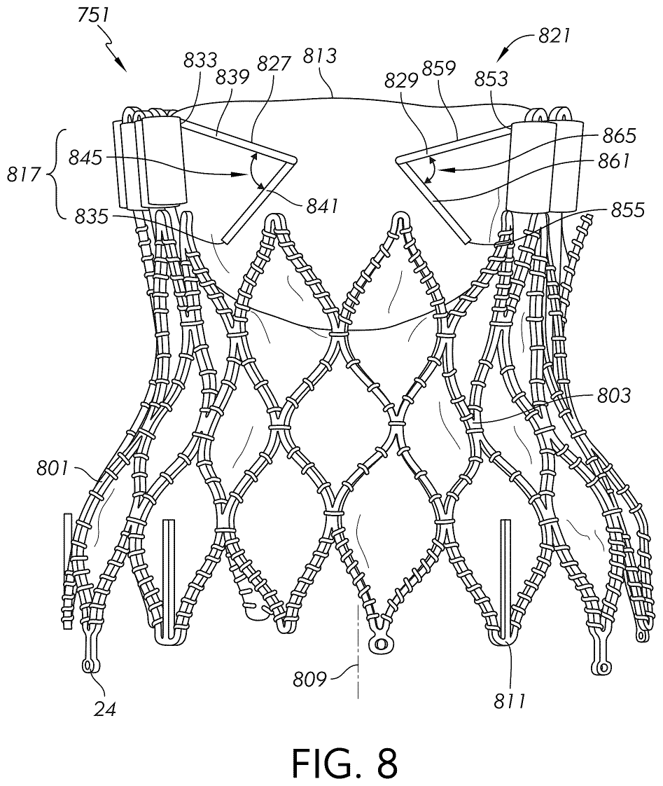

FIG. 8 illustrates a side view of an example of the heart valve prosthesis 751 that can be delivered to the interior of the index heart valve prosthesis 10. For example, the heart valve prosthesis 751 can comprise an annular frame 801 comprising a plurality of frame members or struts 803, with the annular frame 801 configured to be adjustable between a radially-collapsed configuration (e.g., illustrated in FIG. 7) and a radially-expanded configuration (e.g., illustrated in FIG. 8). The frame 801 can be similar to the frame 15 described herein, with the frame 801 supporting a prosthetic valve (e.g., substantially identical to the prosthetic valve 20 comprising at least one leaflet 21 disposed within and secured to the frame 801) within an interior of the frame 801. In some aspects, the frame 801 can comprise a nitinol material. The frame 801 can be self-expandable, balloon-expandable, or mechanically expandable in other embodiments. The frame 801 can extend along a valve axis 809 between a first valve end 811 and a second valve end 813. The first valve end 811 forms the inflow end and the second valve end 813 forms the outflow end. In aspects, the first valve end 811 and the second valve end 813 may comprise some differences from the index valve prosthesis 10. For example, the first valve end 811 can comprise attachment members 24 and retention anchors. Further, the second valve end 813 can comprise fewer coronary access crowns that are wider and do not flare as much as the crowns at the inflow end 11 of the index valve prosthesis 10.

In aspects, the heart valve prosthesis 751 can comprise at least one attachment member 24 attached to the first valve end 811 and extending longitudinally from the first valve end 811 parallel to the valve axis 809. The at least one attachment member 24 can comprise, for example, paddles that can be received within a portion of the valve delivery assembly 30, for example, the spindle 38. In aspects, the spindle 38 can be positioned adjacent to the first valve end 811, and the spindle 38 can comprise one or more spindle pockets (e.g., openings, recesses, cavities, etc.), wherein the at least one attachment member 24 can be received within the spindle pockets. In this way, the at least one attachment member 24 and the spindle can function to maintain the first valve end 811 in the radially-compressed configuration, at least until the at least one attachment member 24 has been removed from the spindle pockets.

The heart valve prosthesis 751 can comprise an engagement region 817 extending circumferentially around the valve axis 809 and located axially between the first valve end 811 and the second valve end 813. In aspects, the engagement region 817 can be located axially between the first valve end 811 and the second valve end 813 such that the engagement region is located an axial distance from the first valve end 811 and the second valve end 813. That is, as illustrated in FIG. 8, the engagement region 817 can be located a first axial distance from the first valve end 811, and the engagement region 817 can be located a second axial distance from the second valve end 813.

The engagement region 817 can comprise at least one protrusion arm 821 extending radially outwardly from the annular frame 801. For example, the at least one protrusion arm 821 can comprise a first protrusion arm 827, a second protrusion arm 829, and, in aspects, zero or more additional protrusion arms that are located radially exterior of the frame 801. The second protrusion arm 829 (e.g., and other protrusion arms) may be similar or identical in structure to the first protrusion arm 827. In aspects, the protrusion arms 821, 827, 829 can comprise a self-expanding material that is biased to radially-expand, such as, for example, nitinol. As used herein, the self-expanding material can be heat-set such that the self-expanding material is biased to move to a radially-expanded position. In this way, the protrusion arms 827, 829 can move between a compressed position (e.g., with each protrusion arm 827, 829 extending substantially linearly) and an expanded position (e.g., illustrated in FIG. 8) in which each protrusion arm 827, 829 can extend non-linearly. The protrusion arms 827, 829 can be biased or heat-set to move to the radially-expanded, non-linear position illustrated in FIG. 8.

With reference to FIG. 8, the first protrusion arm 827 can extend non-linearly between a first arm end 833 and an opposing second arm end 835. The first arm end 833 can be attached to one of the commissures 25, and the second arm end 835 can be spaced apart from, and not in contact with, the plurality of frame members 803. In aspects, the first arm end 833 and the second arm end 835 can be located at different longitudinal locations along the valve axis 809 but at similar circumferential locations about the valve axis 809. As used herein, by being attached to, the first arm end 833 can be separately connected to (e.g., by riveting), or alternatively, formed together (e.g., one-piece formed as a single unitary structure) with a commissure or frame member 803. In this way, the first protrusion arm 827 can project radially outwardly from the frame 801. In addition, in aspects, the first protrusion arm 827 can comprise a bent, non-linear shape that projects from one valve end toward an opposing valve end. In this way, the first protrusion arm 827 may be located a non-constant distance from the first valve end 811. For example, the first arm end 833 can represent a maximum separating distance between the first protrusion arm 827 and the first valve end 811 along the valve axis 809, while the second arm end 835 can represent a minimum separating distance between the first protrusion arm 827 and the first valve end 811 along the valve axis 809.

In aspects, the first protrusion arm 827 can comprise a plurality of arm segments, such as, for example, a first arm segment 839 and a second arm segment 841. The first arm segment 839 and the second arm segment 841 can be attached to one another, with the first arm segment 839 comprising the first arm end 833, and the second arm segment 841 comprising the second arm end 835. The first arm segment 839 can extend non-linearly relative to the second arm segment 841, for example, with an arm angle 845 defined between the first arm segment 839 and the second arm segment 841. In aspects, the arm angle 845 may be within a range from about 60 degrees to about 120 degrees, or between about 75 degrees to about 105 degrees. In aspects, the first arm segment 839 can extend substantially linearly between the first arm end 833 and an end of the first arm segment 839 that is attached to the second arm segment 841. Similarly, in aspects, the second arm segment 841 can extend substantially linearly between the second arm end 835 and an end of the second arm segment 841 that is attached to the first arm segment 839. In this way, beginning at the first arm end 833, the first arm segment 839 can project toward the second protrusion arm 829, while the second arm segment 841, beginning at the attachment to the first arm segment 839, can project away from the second protrusion arm 829. The second arm end 835 can be spaced apart from the plurality of frame members 803 and can contact at least one lacerated leaflet of the index valve prosthesis 10 and move the at least one lacerated leaflet to a position that does not obstruct a coronary artery.

In aspects, the second protrusion arm 829 may be substantially identical in size, shape, structure, and function to the first protrusion arm 827. However, the second protrusion arm 829 can be circumferentially offset from the first protrusion arm 827 about the valve axis 809, for example, within a range from about 100 degrees to about 140 degrees, or about 120 degrees. Likewise, the second protrusion arm 829 can be circumferentially offset from other protrusion arms about the valve axis 809, for example, within a range from about 100 degrees to about 140 degrees, or about 120 degrees.

The second protrusion arm 829 can extend non-linearly between a first arm end 853 and an opposing second arm end 855. The first arm end 853 can be attached to one of the commissures 25, and the second arm end 855 can be spaced apart from, and not in contact with, the plurality of frame members 803. In aspects, the first arm end 853 and the second arm end 855 can be located at different longitudinal locations along the valve axis 809 but at similar circumferential locations about the valve axis 809. The second protrusion arm 829 can project radially outwardly from the frame 801. In addition, in aspects, the second protrusion arm 829 can comprise a bent, non-linear shape that projects from one valve end toward an opposing valve end. In this way, the second protrusion arm 829 may be located a non-constant distance from the first valve end 811. For example, the first arm end 853 can represent a maximum separating distance between the second protrusion arm 829 and the first valve end 811 along the valve axis 809, while the second arm end 855 can represent a minimum separating distance between the second protrusion arm 829 and the first valve end 811 along the valve axis 809.

In aspects, the second protrusion arm 829 can comprise a plurality of arm segments, such as, for example, a third arm segment 859 and a fourth arm segment 861. The third arm segment 859 and the fourth arm segment 861 can be attached to one another, with the third arm segment 859 comprising the first arm end 853, and the fourth arm segment 861 comprising the second arm end 855. The third arm segment 859 can extend non-linearly relative to the fourth arm segment 861, for example, with an arm angle 865 defined between the third arm segment 859 and the fourth arm segment 861. In aspects, the arm angle 865 may be within a range from about 60 degrees to about 120 degrees, or between about 75 degrees to about 105 degrees. In aspects, the third arm segment 859 can extend substantially linearly between the first arm end 853 and an end of the third arm segment 859 that is attached to the fourth arm segment 861. Similarly, in aspects, the fourth arm segment 861 can extend substantially linearly between the second arm end 855 and an end of the fourth arm segment 861 that is attached to the third arm segment 859. In this way, beginning at the first arm end 853, the third arm segment 859 can project toward the first protrusion arm 827, while the fourth arm segment 861, beginning at the attachment to the third arm segment 859, can project away from the first protrusion arm 827. The second arm end 855 can be spaced apart from the plurality of frame members 803 and can contact at least one lacerated leaflet of the index valve prosthesis 10 and move the at least one lacerated leaflet to a position that does not obstruct a coronary artery.

Together, the first protrusion arm 827 and the second protrusion arm 829 can contact at least one lacerated leaflet of the index valve prosthesis 10 and move the at least one lacerated leaflet to a position that does not obstruct a coronary artery. For example, the second arm end 835 of the first protrusion arm 827 can contact at least one lacerated leaflet and move the at least one lacerated leaflet. Similarly, the second arm end 855 of the second protrusion arm 829 can contact at least one lacerated leaflet and move the at least one lacerated leaflet.

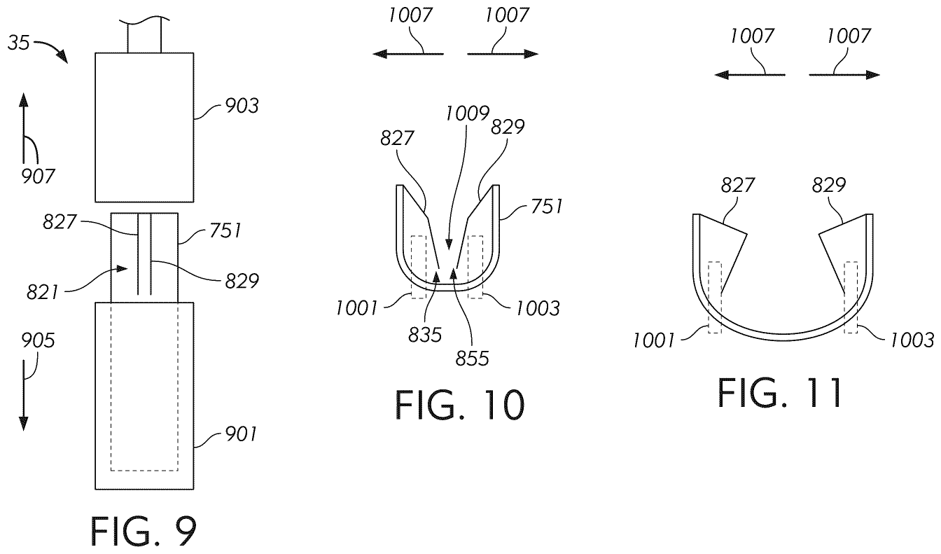

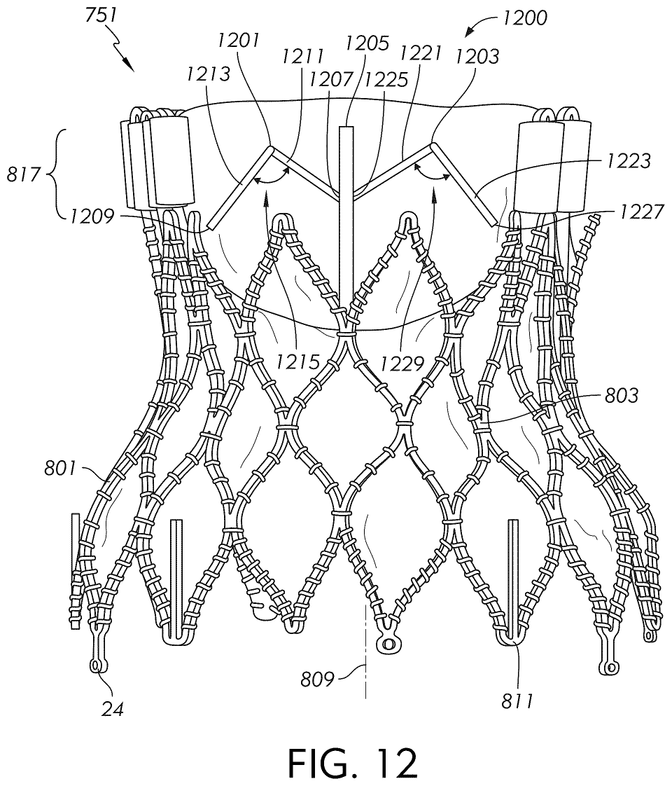

FIGS. 9-11 illustrate example aspects of deploying the heart valve prosthesis 751 within the interior index lumen of the index heart valve prosthesis 10. Initially, methods can comprise lacerating index leaflets 21 of the index valve prosthesis 10 that is positioned in the native aortic annulus 703. The leaflets 21 are lacerated prior to the deployment of the heart valve prosthesis 751. Following the laceration of the leaflets 21, methods can comprise delivering the heart valve prosthesis 751 to the interior index lumen of the heart valve prosthesis 10. In aspects, the heart valve prosthesis 751 can be delivered in a similar manner as described relative to FIGS. 3-7. For example, and as illustrated in FIGS. 7 and 12, the heart valve prosthesis 751 can be radially-compressed and loaded into the capsule 35. That is, the heart valve prosthesis 751 is in the radially-compressed configuration in FIGS. 7 and 9. The capsule 35 can be moved through a patient's vasculature to the native aortic annulus 703. In this way, delivering the heart valve prosthesis 751 can comprise holding the heart valve prosthesis 751 in the radially-compressed configuration within the capsule 35 as the heart valve prosthesis 751 is moved to the interior index lumen of the index heart valve prosthesis 10. As used herein, when the heart valve prosthesis 751 is radially-compressed, the heart valve prosthesis 751 comprises a diameter that is less than the diameter of the heart valve prosthesis 751 when the heart valve prosthesis 751 is expanded and released from the capsule 35 (e.g., illustrated in FIG. 8).

Referring to FIG. 9, to deploy the heart valve prosthesis 751, the capsule 35 can be separated. For example, in some aspects, the capsule 35 can comprise a first sheath 901 and a second sheath 903 that, together, circumferentially surround the heart valve prosthesis 751 and maintain the heart valve prosthesis 751 in the radially-compressed configuration. In aspects, the first valve end 811 can be positioned within the first sheath 901, while the protrusion arms 821 and the second valve end 813 can be positioned within the second sheath 903. To deploy the heart valve prosthesis 751, the first sheath 901 and/or the second sheath 903 can be moved apart from one another, for example, with the first sheath 901 moved in a movement direction 905 away from the second sheath 903 and/or the second sheath 903 moved in a movement direction 907 away from the first sheath 901. Initially, during deployment, the heart valve prosthesis 751 and the second sheath 903 can be moved relative to one another such that the second valve end 813 and the protrusion arms 821 can be removed from the second sheath 903. In this way, the first valve end 811 can, initially, remain within the first sheath 901 while the protrusion arms 821 are uncovered and not within the sheaths 901, 903 of the capsule 35. Due to the protrusion arms 821 comprising a self-expanding material (e.g., nitinol, for example) that is biased to move to the expanded position illustrated in FIG. 8, the protrusion arms 821 can move from a compressed position (e.g., illustrated in FIG. 9) to an expanded position upon being released from the second sheath 903. As can be seen in FIG. 9, the proximal end (e.g., second valve end 813) of the heart valve prosthesis 751 and the protrusion arms 821, can be exposed first prior to releasing and exposing the distal end (e.g., first valve end 811).

FIG. 10 illustrates an enlarged view of a portion of the heart valve prosthesis 751 (e.g., the second valve end 813 and the protrusion arms 821) positioned relative to example leaflet portions 1001, 1003. In aspects, upon being released from the second sheath 903, the protrusion arms 821 can contact the leaflet portions 1001, 1003 and begin to radially expand. For example, lacerating the leaflets can form a first leaflet portion 1001 and a second leaflet portion 1003. For purposes of illustration and to not obstruct the leaflet portions 1001, 1003 from view, the other portions of the index valve prosthesis 10 are omitted from view in FIG. 10. Likewise, while two protrusion arms 821 are illustrated in FIG. 10, the other portions of the heart valve prosthesis 751 are omitted from view. The leaflet(s) can be lacerated to form the leaflet portions 1001, 1003 prior to being engaged by the protrusion arms 821.

As illustrated in FIG. 10, the first protrusion arm 827 can contact and engage the first leaflet portion 1001 while the second protrusion arm 829 can contact and engage the second leaflet portion 1003. This engagement, for example, by the second arm ends 835, 855 of the protrusion arms 821 can allow the protrusion arms 821 to grip the leaflet portions 1001, 1003 and, in aspects, penetrate or extend through the leaflet portions 1001, 1003. Further, the first protrusion arm 827 and the second protrusion arm 829 can bend (e.g., due to being biased to a bent shape) to form the non-linear shape illustrated in FIG. 8. In this way, the first protrusion arm 827 and the second protrusion arm 829 can cause the leaflet portions 1001, 1003 to spread apart (e.g., splay) in a separation direction 1007 (e.g., illustrated in FIG. 10). That is, the first leaflet portion 1001 can be pushed away from the second leaflet portion 1003, and the second leaflet portion 1003 can be pushed away from the first leaflet portion 1001. Accordingly, as illustrated in FIG. 10, a gap 1009 can be formed between the first leaflet portion 1001 and the second leaflet portion 1003. The protrusion arms 821 can remain in engagement with the leaflet portions 1001, 1003 and may prevent the leaflet portions 1001, 1003 from moving to a position that obstructs the coronary arteries. In this way, methods can comprise contacting the lacerated index leaflet(s) 1001, 1003 of the index valve 10 with the protrusion arms 821 such that the lacerated index leaflet(s) 1001, 1003 can be moved to a position that does not obstruct a coronary artery.

Referring to FIG. 11, after the protrusion arms 821 have engaged the leaflet portions 1001, 1003, the heart valve prosthesis 751 can be radially-expanded. In some aspects, the heart valve prosthesis 751 can be balloon-expandable or mechanically-expandable, such that the heart valve prosthesis 751 can be radially expanded with the assistance of a device such as a balloon or other mechanical structure. Alternatively, in aspects, the heart valve prosthesis 751 can comprise a self-expanding material (e.g., nitinol) such that the heart valve prosthesis 751 can radially expand upon being released from the capsule 35. In either of these examples, the radial expansion of the heart valve prosthesis 751 can cause the first protrusion arm 827 and the second protrusion arm 829 to move away from one another, thus increasing a separating distance between the first protrusion arm 827 and the second protrusion arm 829. As the first protrusion arm 827 and the second protrusion arm 829 move apart, the first protrusion arm 827 and the second protrusion arm 829 can cause the leaflet portions 1001, 1003 to continue to spread apart (e.g., splay) in the separation direction 1007.

FIGS. 12-14 illustrate additional embodiments of the protrusion arm. For example, referring to FIG. 12, the heart valve prosthesis 751 can comprise at least one protrusion arm 1200 that is different in structure, but similar in function, to the protrusion arms 821 of FIGS. 8-11. For example, the at least one protrusion arm 1200 can extend radially outwardly from the annular frame 801. The at least one protrusion arm 1200 can comprise a first protrusion arm 1201, a second protrusion arm 1203, and, in aspects, zero or more additional protrusion arms that are located radially exterior of the frame 801. The second protrusion arm 1203 (e.g., and other protrusion arms) may be similar or identical in structure to the first protrusion arm 1201. In aspects, the protrusion arms 1200, 1201, 1203 can comprise a self-expanding material that is biased to radially-expand, such as, for example, nitinol. As used herein, the self-expanding material can be heat-set such that the self-expanding material is biased to move to a radially-expanded position.

With reference to FIG. 12, the first protrusion arm 1201 can extend non-linearly between a first arm end 1207 and an opposing second arm end 1209. The first arm end 1207 can be attached to an attachment post 1205 of the frame 801. In aspects, the attachment post 1205 can extend substantially linearly and parallel to the valve axis 809, with the attachment post 1205 attached to one or more of the frame members 803. In aspects, the attachment post 1205 can be positioned circumferentially between two of the commissures. The second arm end 1209 can be spaced apart from, and not in contact with, the plurality of frame members 803. In aspects, the first arm end 1207 and the second arm end 1209 can be located at the same, or different, longitudinal locations along the valve axis 809 but at different circumferential locations about the valve axis 809. The first protrusion arm 1201 can project radially outwardly from the frame 801. In addition, in aspects, the first protrusion arm 1201 can comprise a bent, non-linear shape that projects away from the attachment post 1205. In this way, the first protrusion arm 1201 may be located a non-constant distance from the first valve end 811. For example, the first arm end 1207 can represent a first separating distance between the first protrusion arm 1201 and the first valve end 811 along the valve axis 809, while a midpoint of the first protrusion arm 1201 (e.g., between the first arm end 1207 and the second arm end 1209) can represent a differing separating distance between the first protrusion arm 1201 and the first valve end 811 along the valve axis 809.

In aspects, the first protrusion arm 1201 can comprise a plurality of arm segments, such as, for example, a first arm segment 1211 and a second arm segment 1213. The first arm segment 1211 and the second arm segment 1213 can be attached to one another, with the first arm segment 1211 comprising the first arm end 1207, and the second arm segment 1213 comprising the second arm end 1209. The first arm segment 1211 can extend non-linearly relative to the second arm segment 1213, for example, with an arm angle 1215 defined between the first arm segment 1211 and the second arm segment 1213. In aspects, the arm angle 1215 may be within a range from about 60 degrees to about 120 degrees, or between about 75 degrees to about 105 degrees. In aspects, the first arm segment 1211 can extend substantially linearly between the first arm end 1207 and an end of the first arm segment 1211 that is attached to the second arm segment 1213. Similarly, in aspects, the second arm segment 1213 can extend substantially linearly between the second arm end 1209 and an end of the second arm segment 1213 that is attached to the first arm segment 1211. In this way, beginning at the first arm end 1207, the first arm segment 1211 can project away from the attachment post 1205, while the second arm segment 1213, beginning at the attachment to the first arm segment 1211, can project away from the attachment post 1205. The second arm end 1209 can be spaced apart from the plurality of frame members 803 and can contact at least one lacerated leaflet of the index valve prosthesis 10 and move the at least one lacerated leaflet to a position that does not obstruct a coronary artery.

In aspects, the second protrusion arm 1203 may be substantially identical in size, shape, structure, and function to the first protrusion arm 1201. However, the second protrusion arm 1203 can be circumferentially offset from the first protrusion arm 1201 about the valve axis 809. For example, the second protrusion arm 1203 can be attached to the attachment post 1205 on an opposite side of the attachment post 1205 from the first protrusion arm 1201. The second protrusion arm 1203 can extend non-linearly between a first arm end 1225 and an opposing second arm end 1227. The first arm end 1225 can be attached to the attachment post 1205, and the second arm end 1227 can be spaced apart from, and not in contact with, the plurality of frame members 803. In aspects, the first arm end 1225 and the second arm end 1227 can be located at the same, or different, longitudinal locations along the valve axis 809 but at different circumferential locations about the valve axis 809. The second protrusion arm 1203 can project radially outwardly from the frame 801. In addition, in aspects, the second protrusion arm 1203 can comprise a bent, non-linear shape that projects away from the attachment post 1205. In this way, the second protrusion arm 1203 may be located a non-constant distance from the first valve end 811. For example, the first arm end 1225 can represent a first separating distance between the second protrusion arm 1203 and the first valve end 811 along the valve axis 809, while a midpoint of the second protrusion arm 1203 (e.g., between the first arm end 1225 and the second arm end 1227) can represent a differing separating distance between the second protrusion arm 1203 and the first valve end 811 along the valve axis 809.

In aspects, the second protrusion arm 1203 can comprise a plurality of arm segments, such as, for example, a third arm segment 1221 and a fourth arm segment 1223. The third arm segment 1221 and the fourth arm segment 1223 can be attached to one another, with the third arm segment 1221 comprising the first arm end 1225, and the fourth arm segment 1223 comprising the second arm end 1227. The third arm segment 1221 can extend non-linearly relative to the fourth arm segment 1223, for example, with an arm angle 1229 defined between the third arm segment 1221 and the fourth arm segment 1223. In aspects, the arm angle 1229 may be within a range from about 60 degrees to about 120 degrees, or between about 75 degrees to about 105 degrees. In aspects, the third arm segment 1221 can extend substantially linearly between the first arm end 1225 and an end of the third arm segment 1221 that is attached to the fourth arm segment 1223. Similarly, in aspects, the fourth arm segment 1223 can extend substantially linearly between the second arm end 1227 and an end of the fourth arm segment 1223 that is attached to the third arm segment 1221. In this way, beginning at the first arm end 1225, the third arm segment 1221 can project away from the attachment post 1205, while the fourth arm segment 1223, beginning at the attachment to the third arm segment 1221, can project away from the first protrusion arm 1201. The second arm end 1227 can be spaced apart from the plurality of frame members 803 and can contact at least one lacerated leaflet of the index valve prosthesis 10 and move the at least one lacerated leaflet to a position that does not obstruct a coronary artery.

Together, the first protrusion arm 1201 and the second protrusion arm 1203 can contact at least one lacerated leaflet of the index valve prosthesis 10 and move the at least one lacerated leaflet to a position that does not obstruct a coronary artery. For example, the second arm end 1209 of the first protrusion arm 1201 can contact at least one lacerated leaflet and move the at least one lacerated leaflet. Similarly, the second arm end 1227 of the second protrusion arm 1203 can contact at least one lacerated leaflet and move the at least one lacerated leaflet. While FIG. 12 illustrates a single pair of protrusion arms 1201, 1203, the heart valve prosthesis 751 is not so limited, and may comprise any number (e.g., one or more) of protrusion arms depending on the number of lacerated leaflets. In this way, when the heart valve prosthesis 751 comprises three pairs of protrusion arms, each pair of protrusion arms may be equally distributed and spaced apart about the circumference of the heart valve prosthesis 751.

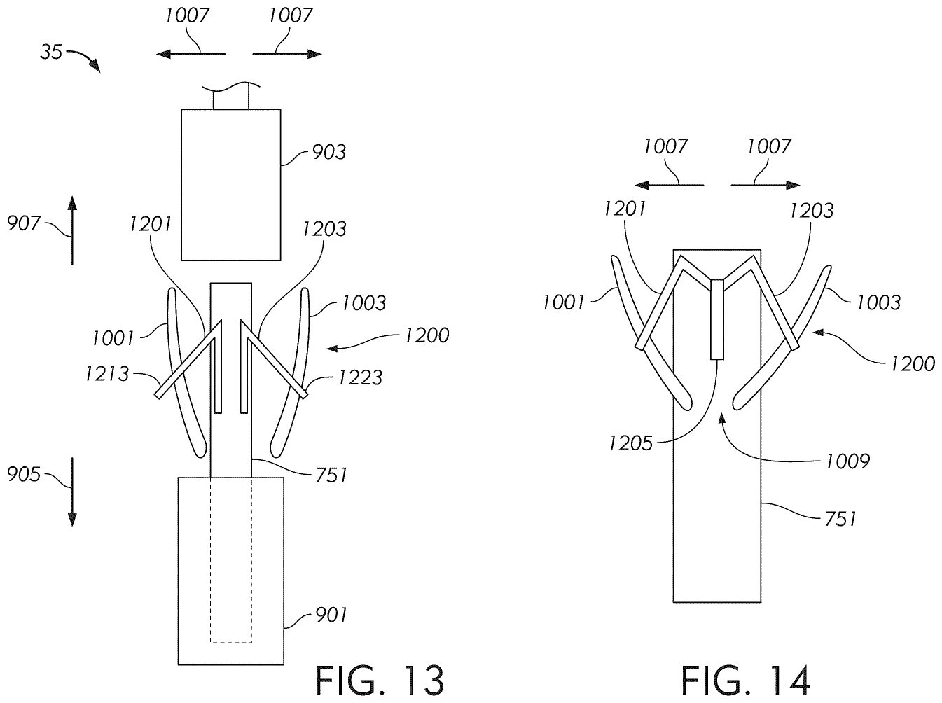

FIGS. 13-14 illustrate example aspects of deploying the heart valve prosthesis 751 within the interior index lumen of the index heart valve prosthesis 10. Initially, methods can comprise lacerating index leaflets 21 of the index valve prosthesis 10 that is positioned in the native aortic annulus 703. In aspects, the heart valve prosthesis 751 can be delivered in a similar manner as described relative to FIGS. 3-7 and 9-11. For example, and as illustrated in FIGS. 7 and 12, the heart valve prosthesis 751 can be radially-compressed and loaded into the capsule 35. That is, similar to FIGS. 7 and 9, the heart valve prosthesis 751 is in the radially-compressed configuration in FIG. 13. The capsule 35 in FIG. 13, and methods of delivering the capsule 35 through the patient's vasculature to the native aortic annulus 703, is substantially identical to the capsule 35 and delivery methods described above.

Referring to FIG. 13, the heart valve prosthesis 751 can be deployed in a similar manner as described relative to FIGS. 9-11. For example, the capsule 35 can comprise the first sheath 901 and the second sheath 903 that, together, circumferentially surround the heart valve prosthesis 751 and maintain the heart valve prosthesis 751 in the radially-compressed configuration. In aspects, the first valve end 811 can be positioned within the first sheath 901, while the protrusion arms 1200 and the second valve end 813 can be positioned within the second sheath 903. To deploy the heart valve prosthesis 751, the first sheath 901 and/or the second sheath 903 can be moved apart from one another, for example, with the first sheath 901 moved in the movement direction 905 away from the second sheath 903 and/or the second sheath 903 moved in the movement direction 907 away from the first sheath 901. Due to the protrusion arms 1200 comprising a self-expanding material (e.g., nitinol, for example) that is biased to move to the expanded position illustrated in FIG. 12, the protrusion arms 1200 can move from a compressed position (e.g., illustrated in FIG. 13) to an expanded position upon being released from the second sheath 903. Alternatively, the protrusion arms 1200 may not comprise a self-expanding material, but, instead, may be deployed separately by a portion of the delivery apparatus. As can be seen in FIG. 13, the proximal end (e.g., second valve end 813) of the heart valve prosthesis 751 and the protrusion arms 1200, can be exposed first prior to releasing and exposing the distal end (e.g., first valve end 811).

FIGS. 13-14 illustrate the heart valve prosthesis 751 (e.g., the second valve end 813 and the protrusion arms 1200) positioned relative to the leaflet portions 1001, 1003. In aspects, upon being released from the second sheath 903, the protrusion arms 1200 can contact the leaflet portions 1001, 1003 and begin to radially expand. As illustrated in FIG. 14, the first protrusion arm 1201 can contact and engage the first leaflet portion 1001 while the second protrusion arm 1203 can contact and engage the second leaflet portion 1003. This engagement, for example, by the second arm ends 1209, 1227 of the protrusion arms 1200 can allow the protrusion arms 1200 to grip the leaflet portions 1001, 1003 and, in aspects, penetrate or extend through the leaflet portions 1001, 1003. In this way, the first protrusion arm 1201 and the second protrusion arm 1203 can cause the leaflet portions 1001, 1003 to spread apart (e.g., splay) in the separation direction 1007. That is, the first leaflet portion 1001 can be pushed away from the second leaflet portion 1003, and the second leaflet portion 1003 can be pushed away from the first leaflet portion 1001. Accordingly, as illustrated in FIG. 14, the gap 1009 can be formed between the first leaflet portion 1001 and the second leaflet portion 1003. The protrusion arms 1200 can remain in engagement with the leaflet portions 1001, 1003 and may prevent the leaflet portions 1001, 1003 from moving to a position that obstructs the coronary arteries. In this way, methods can comprise contacting the lacerated index leaflet(s) 1001, 1003 of the index valve 10 with the protrusion arms 1200 such that the lacerated index leaflet(s) 1001, 1003 can be moved to a position that does not obstruct a coronary artery.

Referring to FIG. 14, after the protrusion arms 1200 have engaged the leaflet portions 1001, 1003, the heart valve prosthesis 751 can be radially-expanded. As with the previous examples, the heart valve prosthesis 751 can be balloon-expandable or mechanically-expandable, such that the heart valve prosthesis 751 can be radially expanded with the assistance of a device such as a balloon or other mechanical structure. Alternatively, in aspects, the heart valve prosthesis 751 can comprise a self-expanding material (e.g., nitinol) such that the heart valve prosthesis 751 can radially expand upon being released from the capsule 35. In either of these examples, the radial expansion of the heart valve prosthesis 751 can cause the first protrusion arm 1201 and the second protrusion arm 1203 to move away from one another, thus increasing a separating distance between the first protrusion arm 1201 and the second protrusion arm 1203. As the first protrusion arm 1201 and the second protrusion arm 1203 move apart, the first protrusion arm 1201 and the second protrusion arm 1203 can cause the leaflet portions 1001, 1003 to continue to spread apart (e.g., splay) in the separation direction 1007.

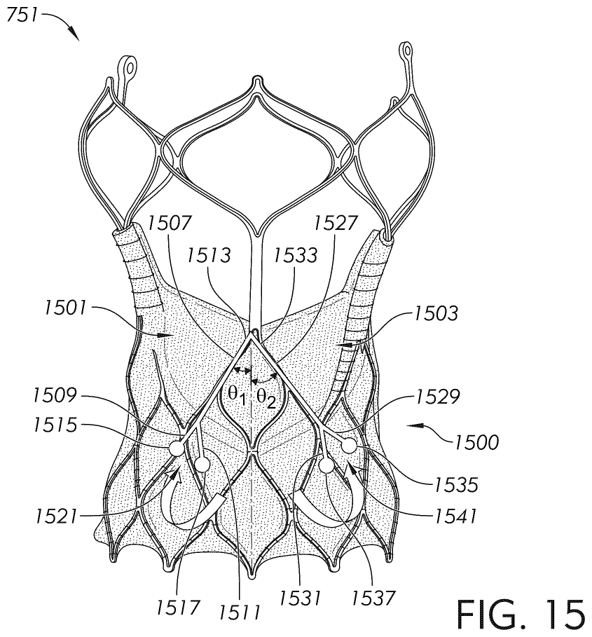

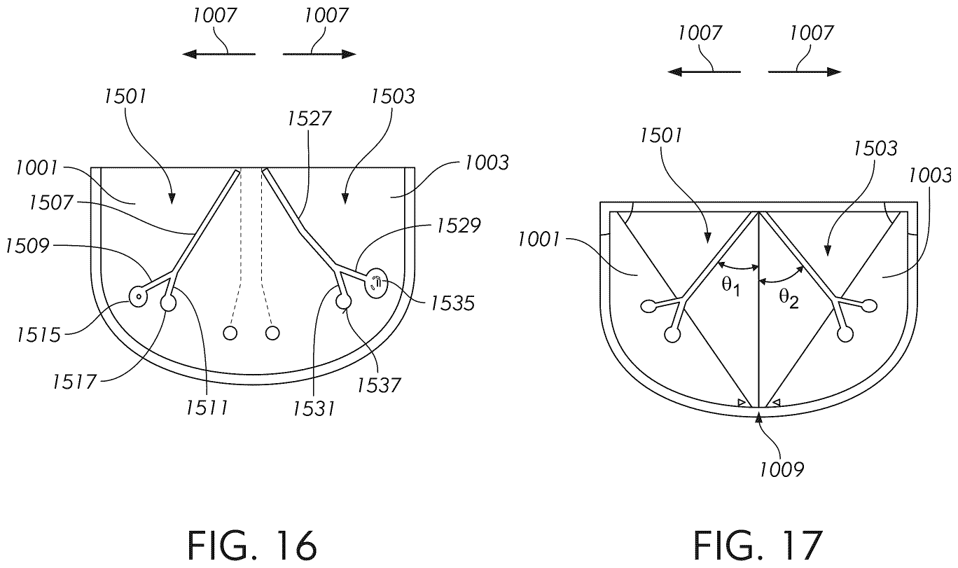

FIGS. 15-16 illustrate additional embodiments of the protrusion arm. For example, referring to FIG. 15, the heart valve prosthesis 751 can comprise at least one protrusion arm 1500 that is different in structure, but similar in function, to the protrusion arms 821, 1200 described herein. For example, the at least one protrusion arm 1500 can extend radially outwardly from the annular frame 801. The at least one protrusion arm 1500 can comprise a first protrusion arm 1501, a second protrusion arm 1503, and, in aspects, zero or more additional protrusion arms that are located radially exterior of the frame 801. The second protrusion arm 1503 (e.g., and other protrusion arms) may be similar or identical in structure to the first protrusion arm 1501. In aspects, the protrusion arms 1500, 1501, 1503 can comprise a self-expanding material that is biased to radially-expand, such as, for example, nitinol. As used herein, the self-expanding material can be heat-set such that the self-expanding material is biased to move to a radially-expanded position.

With reference to FIG. 15, the first protrusion arm 1501 can extend non-linearly between a first arm end 1513 and an opposing second arm end 1515 and a third arm end 1517. The first arm end 1513 can be attached to one or more of the plurality of frame members 803 of the frame 801. The first protrusion arm 1501 can project radially outwardly from the frame 801. In addition, in aspects, the first protrusion arm 1501 can comprise a bent, non-linear shape. In aspects, the first protrusion arm 1501 can comprise a plurality of arm segments, such as, for example, a first arm segment 1507, a second arm segment 1509, and a third arm segment 1511. The first arm segment 1507 can be attached to the second arm segment 1509 and the third arm segment 1511, with the first arm segment 1507 comprising the first arm end 1513. In aspects, the first arm segment 1507 can be attached at one end (e.g., the first arm end 1513) to the frame 801, while an opposing end of the first arm segment 1507 can be attached to the second arm segment 1509 and the third arm segment 1511. In aspects, the first arm segment 1507 can extend substantially linearly from the first arm end 1513.

The second arm segment 1509 and the third arm segment 1511 can be attached to an end of the first arm segment 1507, for example, with the second arm segment 1509 and the third arm segment 1511 attached to a common point or a common end of the first arm segment 1507. In aspects, the first arm segment 1507 can extend non-linearly relative to the second arm segment 1509, for example, with an arm angle defined between the first arm segment 1507 and the second arm segment 1509. Likewise, in aspects, the first arm segment 1507 can extend non-linearly relative to the third arm segment 1511, for example, with an arm angle defined between the first arm segment 1507 and the third arm segment 1511. In aspects, an arm angle 1521 can be defined between the second arm segment 1509 and the third arm segment 1511. The arm angle 1521 may be within a range from about 60 degrees to about 120 degrees, or between about 75 degrees to about 105 degrees. The second arm end 1515 can be defined at an end of the second arm segment 1509, and the third arm end 1517 can be defined at an end of the third arm segment 1511. The second arm end 1515 and the third arm end 1517 can be spaced apart from one another and can contact at least one lacerated leaflet of the index valve prosthesis 10 and move the at least one lacerated leaflet to a position that does not obstruct a coronary artery.

In aspects, the second protrusion arm 1503 may be substantially identical in size, shape, structure, and function to the first protrusion arm 1501. However, the second protrusion arm 1503 can be circumferentially offset from the first protrusion arm 1501 about the valve axis 809. The second protrusion arm 1503 can extend non-linearly between a first arm end 1533 and an opposing second arm end 1535 and a third arm end 1537. The first arm end 1533 can be attached to one or more of the plurality of frame members 803 of the frame 801. The first protrusion arm 1501 can project radially outwardly from the frame 801. In addition, in aspects, the second protrusion arm 1503 can comprise a bent, non-linear shape. In aspects, the second protrusion arm 1503 can comprise a plurality of arm segments, such as, for example, a first arm segment 1527, a second arm segment 1529, and a third arm segment 1531. The first arm segment 1527 can be attached to the second arm segment 1529 and the third arm segment 1531, with the first arm segment 1527 comprising the first arm end 1533. In aspects, the first arm segment 1527 can be attached at one end (e.g., the first arm end 1533) to the frame 801, while an opposing end of the first arm segment 1527 can be attached to the second arm segment 1529 and the third arm segment 1531. In aspects, the first arm segment 1527 can extend substantially linearly from the first arm end 1533.

The second arm segment 1529 and the third arm segment 1531 can be attached to an end of the first arm segment 1527, for example, with the second arm segment 1529 and the third arm segment 1531 attached to a common point or a common end of the first arm segment 1527. In aspects, the first arm segment 1527 can extend non-linearly relative to the second arm segment 1529, for example, with an arm angle defined between the first arm segment 1527 and the second arm segment 1529. Likewise, in aspects, the first arm segment 1527 can extend non-linearly relative to the third arm segment 1531, for example, with an arm angle defined between the first arm segment 1527 and the third arm segment 1531. In aspects, an arm angle 1541 can be defined between the second arm segment 1529 and the third arm segment 1531. The arm angle 1541 may be within a range from about 60 degrees to about 120 degrees, or between about 75 degrees to about 105 degrees. The second arm end 1535 can be defined at an end of the second arm segment 1529, and the third arm end 1537 can be defined at an end of the third arm segment 1531. The second arm end 1535 and the third arm end 1537 can be spaced apart from one another and can contact at least one lacerated leaflet of the index valve prosthesis 10 and move the at least one lacerated leaflet to a position that does not obstruct a coronary artery.

Together, the first protrusion arm 1501 and the second protrusion arm 1503 can contact at least one lacerated leaflet of the index valve prosthesis 10 and move the at least one lacerated leaflet to a position that does not obstruct a coronary artery. For example, the second arm end 1515 and the third arm end 1517 of the first protrusion arm 1501 can contact at least one lacerated leaflet and move the at least one lacerated leaflet. Similarly, the second arm end 1535 and the third arm end 1537 of the second protrusion arm 1503 can contact at least one lacerated leaflet and move the at least one lacerated leaflet. In aspects, the protrusion arms 1501, 1503 can be located below the margin of attachment (MOA) of the heart valve prosthesis 751 to reduce the likelihood of damage to the heart valve prosthesis 751 when the heart valve prosthesis 751 is in the radially-compressed, or crimped, position. In aspects, the protrusion arms 1501, 1503 may be nearly flush with an outer diameter of the valve prosthesis 751 to push the leaflet portions against the anatomy. In aspects, the first protrusion arm 1501 may project at an angle θ₁ and the second protrusion arm 1503 may project at an angle θ₂ (FIGS. 15 and 17) relative to a centerline extending between the first protrusion arm 1501 and the second protrusion arm 1503. In aspects, the angles θ₁ and θ₂ are about 45 degrees. In aspects, the angle θ₁ and the angle θ₂ may be the same or different.

FIGS. 16-17 illustrate the protrusion arms 1500 contacting and moving the leaflet portions 1001, 1003. For example, the heart valve prosthesis 751 can be delivered to the interior index lumen of the heart valve prosthesis 10 and deployed in a substantially identical manner as described relative to FIGS. 9-14. That is, the heart valve prosthesis 751 can be radially-compressed and loaded into the capsule 35 prior to being moved through the patient's vasculature. Upon reaching the native aortic annulus 703, the heart valve prosthesis 751 can be partially released from the capsule 35 such that the protrusion arms 1500 can contact and engage the leaflet portions 1001, 1003. As illustrated in FIG. 16, the protrusion arms 1500 can initially be in a radially-compressed configuration (e.g., illustrated with dashed lines) while positioned within the capsule 35. Upon releasing the protrusion arms 1500 from the capsule 35, the protrusion arms 1500 can move from the radially-compressed configuration (e.g., illustrated with dashed lines) to the expanded configuration (e.g., illustrated with solid lines in FIG. 16). In aspects, in the expanded configurations, the first protrusion arm 1501 may project at the angle θ₁ and the second protrusion arm 1503 may project at the angle θ₂ relative to the centerline extending between the first protrusion arm 1501 and the second protrusion arm 1503. In aspects, the angles θ₁ and θ₂ are about 45 degrees. In aspects, the angle θ₁ and the angle θ₂ may be the same or different.

As illustrated in FIG. 17, the first protrusion arm 1501 can contact and engage the first leaflet portion 1001 while the second protrusion arm 1503 can contact and engage the second leaflet portion 1003. This engagement, for example, by the second and third arm ends 1515, 1517 of the first protrusion arm 1501 and the second and third arm ends 1535, 1537 of the second protrusion arm 1503 can allow the protrusion arms 1500 to grip the leaflet portions 1001, 1003 and, in aspects, penetrate or extend through the leaflet portions 1001, 1003. In this way, the first protrusion arm 1501 and the second protrusion arm 1503 can cause the leaflet portions 1001, 1003 to spread apart (e.g., splay) in the separation direction 1007. That is, the first leaflet portion 1001 can be pushed away from the second leaflet portion 1003, and the second leaflet portion 1003 can be pushed away from the first leaflet portion 1001. Accordingly, as illustrated in FIG. 17, the gap 1009 can be formed between the first leaflet portion 1001 and the second leaflet portion 1003. The protrusion arms 1500 can remain in engagement with the leaflet portions 1001, 1003 and may prevent the leaflet portions 1001, 1003 from moving to a position that obstructs the coronary arteries. In this way, methods can comprise contacting the lacerated index leaflet(s) 1001, 1003 of the index valve 10 with the protrusion arms 1500 such that the lacerated index leaflet(s) 1001, 1003 can be moved to a position that does not obstruct a coronary artery. In aspects, the arm ends 1515, 1517, 1535, 1537 illustrated in FIG. 15 (e.g., and, in aspects, some, or all, of the other arm ends disclosed herein) can comprise a rounded shape to reduce the likelihood of inadvertent damage to the surrounding tissue. To further reduce the likelihood of inadvertent damage, the arm ends 1515, 1517, 1535, 1537 illustrated in FIG. 15 (e.g., and, in aspects, some, or all, of the other arm ends disclosed herein) can be surrounded or covered by a material comprising one or more of fabric or tissue.

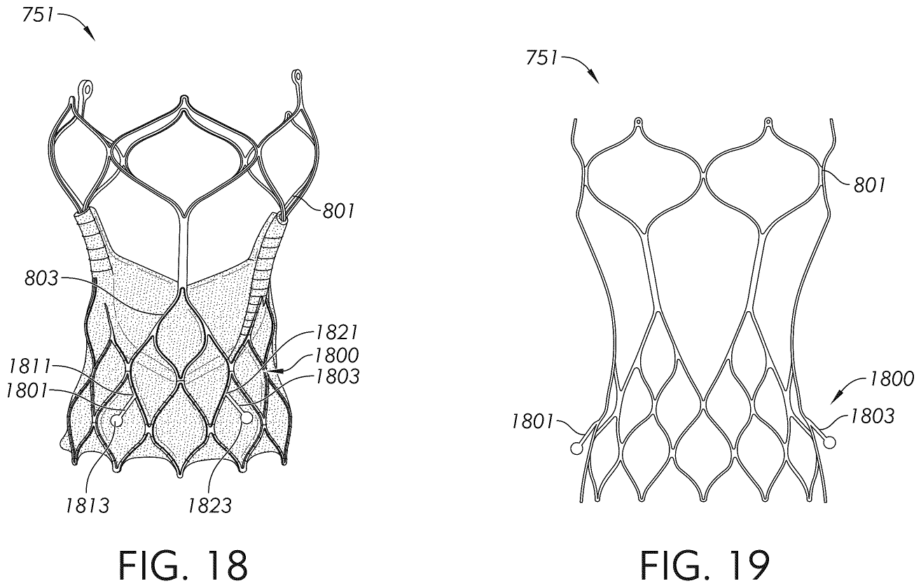

FIGS. 18-19 illustrate additional embodiments of the protrusion arm. For example, referring to FIG. 18, the heart valve prosthesis 751 can comprise at least one protrusion arm 1800 that is different in structure, but similar in function, to the protrusion arms 821, 1200, 1500 described herein. For example, the at least one protrusion arm 1800 can extend radially outwardly from the annular frame 801. The at least one protrusion arm 1800 can comprise a first protrusion arm 1801 and a second protrusion arm 1803, and, in aspects, zero or more additional protrusion arms that are located radially exterior of the frame 801. The second protrusion arm 1803 (e.g., and other protrusion arms) may be similar or identical in structure to the first protrusion arm 1801. In aspects, the protrusion arms 1800, 1801, 1803 can comprise a self-expanding material that is biased to radially-expand, such as, for example, nitinol. As used herein, the self-expanding material can be heat-set such that the self-expanding material is biased to move to a radially-expanded position. In aspects, the protrusion arms 1800 can each be angled at about 45 degrees relative to the valve axis 809.

With reference to FIG. 18, the first protrusion arm 1801 can extend between a first arm end 1811 and an opposing second arm end 1813. The first arm end 1811 can be attached to one or more of the plurality of frame members 803 of the frame 801. The second protrusion arm 1803 can extend between a first arm end 1821 and an opposing second arm end 1823. The first arm end 1821 can be attached to one or more of the plurality of frame members 803 of the frame 801. As illustrated in the side view of FIG. 19, the first protrusion arm 1801 and the second protrusion arm 1803 can project radially outwardly from the frame 801. The protrusion arms 1801, 1803 can function in a substantially identical manner to the protrusion arms 821, 1200, 1500 described above. That is, upon being released from the capsule 35, the protrusion arms 1801, 1803 can contact and engage the leaflet portions 1001, 1003, and cause the leaflet portions 1001, 1003 to spread apart (e.g., splay). As such, the protrusion arms 1801, 1803 can remain in engagement with the leaflet portions 1001, 1003 and may prevent the leaflet portions 1001, 1003 from moving to a position that obstructs the coronary arteries. In this way, methods can comprise contacting the lacerated index leaflet(s) 1001, 1003 of the index valve 10 with the protrusion arms 1801, 1803 such that the lacerated index leaflet(s) 1001, 1003 can be moved to a position that does not obstruct a coronary artery. In aspects, to further facilitate splaying of the lacerated index leaflet(s) 1001, 1003, the heart valve prosthesis 751 can be moved axially along the valve axis 809 while the protrusion arms 1800, 1801, 1803 to push the heart valve prosthesis 751 down and into the leaflet portions 1001, 1003 to facilitate separation of the leaflet portions 1001, 1003.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A heart valve prosthesis configured to be positioned within an index valve prosthesis, the heart valve prosthesis comprising:
an annular frame extending along a valve axis between a first valve end of the heart valve prosthesis and a second valve end of the heart valve prosthesis, the annular frame comprising a plurality of frame members and configured to be adjustable between a radially-collapsed configuration and a radially-expanded configuration, the annular frame comprising:
an engagement region extending circumferentially around the valve axis and comprising:
at least one protrusion arm extending radially outwardly from the annular frame, the at least one protrusion arm comprising a first arm end and an opposing second arm end, the first arm end attached to a frame member of the plurality of frame members, the second arm end spaced apart from the plurality of frame members and configured to contact at least one lacerated leaflet of the index valve prosthesis and move the at least one lacerated leaflet to a position that does not obstruct a coronary artery.

2. The heart valve prosthesis of claim 1, wherein the at least one protrusion arm comprises a first protrusion arm comprising a first arm segment and a second arm segment, the first arm segment comprising the first arm end, and the second arm segment comprising the second arm end.

3. The heart valve prosthesis of claim 2, wherein an arm angle is defined between the first arm segment and the second arm segment, the arm angle within a range from about 60 degrees to about 120 degrees.

4. The heart valve prosthesis of claim 3, wherein the at least one protrusion arm comprises a second protrusion arm that is substantially identical in shape to the first protrusion arm, the second protrusion arm attached to one of the plurality of frame members at substantially the same longitudinal location along the valve axis as the first protrusion arm and at a different circumferential location about the valve axis as the first protrusion arm.

5. The heart valve prosthesis of claim 4, wherein the second protrusion arm comprises a third arm segment and a fourth arm segment, the third arm segment comprising a first arm end attached to the frame member of the plurality of frame members, and the fourth arm segment comprising a fourth arm end opposite the third arm end.

6. The heart valve prosthesis of claim 5, wherein the second protrusion arm is substantially identical in shape to the first protrusion arm, the first protrusion arm and the second protrusion arm attached to one of the plurality of frame members at substantially the same longitudinal location along the valve axis and at substantially the same circumferential location about the valve axis.

7. The heart valve prosthesis of claim 6, wherein the second arm end and the fourth arm end comprise a rounded shape.

8. The heart valve prosthesis of claim 6, wherein the second arm end and the fourth arm end are each surrounded by a material comprising one or more of fabric or tissue.

9. The heart valve prosthesis of claim 1, wherein the at least one protrusion arm comprises a first arm segment, a second arm segment, and a third arm segment, the first arm segment comprising the first arm end, and the second arm segment and the third arm segment are attached to an end of the first arm segment.

10. A heart valve prosthesis configured to be positioned within an index valve prosthesis, the heart valve prosthesis comprising:
an annular frame extending along a valve axis between a first valve end of the heart valve prosthesis and a second valve end of the heart valve prosthesis, the annular frame comprising a plurality of frame members and configured to be adjustable between a radially-collapsed configuration and a radially-expanded configuration, the annular frame comprising:
an engagement region extending circumferentially around the valve axis and comprising:
at least one protrusion arm extending radially outwardly from the annular frame, the at least one protrusion arm extending non-linearly between a first arm end and a second arm end, the first arm end attached to a frame member of the plurality of frame members, the second arm end spaced apart from the plurality of frame members and configured to contact at least one lacerated leaflet of the index valve prosthesis and move the at least one lacerated leaflet to a position that does not obstruct a coronary artery.

11. The heart valve prosthesis of claim 10, wherein the at least one protrusion arm comprises a first protrusion arm comprising a first arm segment and a second arm segment, the first arm segment comprising the first arm end, and the second arm segment comprising the second arm end.

12. The heart valve prosthesis of claim 11, wherein an arm angle is defined between the first arm segment and the second arm segment, the arm angle within a range from about 60 degrees to about 120 degrees.

13. The heart valve prosthesis of claim 12, wherein the at least one protrusion arm comprises a second protrusion arm that is substantially identical in shape to the first protrusion arm, the second protrusion arm attached to one of the plurality of frame members at substantially the same longitudinal location along the valve axis as the first protrusion arm and at a different circumferential location about the valve axis as the first protrusion arm.

14. A method of implanting a heart valve prosthesis comprising:
lacerating index leaflets of an index valve prosthesis that is positioned in a native aortic annulus;
delivering the heart valve prosthesis to an interior index lumen of the index valve prosthesis;
deploying the heart valve prosthesis within the interior index lumen such that the heart valve prosthesis moves from a radially-compressed configuration to a radially-expanded configuration, the heart valve prosthesis comprising at least one protrusion arm extending radially outwardly from an annular frame, the at least one protrusion arm extending non-linearly between a first arm end, which is attached to a frame member of the plurality of frame members, and a second arm end; and
contacting the lacerated index leaflets of the index valve with the second arm end such that the lacerated index leaflets are moved to a position that does not obstruct a coronary artery.

15. The method of claim 14, wherein the second arm end comprises a rounded shape.

16. The method of claim 15, wherein the second arm end is surrounded by a material comprising one or more of fabric or tissue.

17. The method of claim 15, wherein the deploying the heart valve prosthesis comprises moving the at least one protrusion arm from a compressed position to a radially-expanded position.
